(19) United States
(12) Patent Application Publication
(10) Pub. No.: US 2026/0114977 A1
(43) Pub. Date: Apr. 30, 2026

(54) AMBIENT PRESSURE ESTIMATION IN AN IMPLANTABLE MEDICAL DEVICE

(71) Applicant: Boston Scientific Scimed, Inc., Maple Grove, MN (US)

(72) Inventors: Natalie Ann Borgos, Roseville, MN (US); Sean Thomas Horan, Minneapolis, MN (US); Jessica Williams, Marlborough, MA (US); John D. Hatlestad, Maplewood, MN (US); Keith R. Maile, New Brighton, MN (US)

(21) Appl. No.: 19/369,985

(22) Filed: Oct. 27, 2025

Related U.S. Application Data
(60) Provisional application No. 63/713,946, filed on Oct. 30, 2024.

(51) Int. Cl. A61F 2/00; A61F 2/04; A61F 2/48

(52) U.S. Cl. CPC A61F 2/004; A61F 2/04; A61F 2/482; A61F 2/484; A61F 2002/047; A61F 2250/0003; A61F 2250/0013

(57) ABSTRACT

An implantable medical device may include an inflatable member. An implantable medical device may include a fluid reservoir. An implantable medical device may include a pressure sensor connected to the fluid reservoir. An implantable medical device may include an electronic pump device including a controller configured to execute operations, including receiving a signal with pressure readings during a time interval from the pressure sensor, identifying a portion of the pressure readings within a percentile range, computing an ambient pressure value based on the portion of the pressure readings, and updating an ambient pressure based on the ambient pressure value.

### CROSS-REFERENCE TO RELATED APPLICATION

This application claims priority to U.S. Provisional Patent Application No. 63/713,946, filed on Oct. 30, 2024, entitled “AMBIENT PRESSURE ESTIMATION IN AN IMPLANTABLE MEDICAL DEVICE”, the disclosure of which is incorporated by reference herein in its entirety.

### TECHNICAL FIELD

This disclosure relates generally to an implantable medical device for estimating ambient pressure inside of a patient's body to control inflation and/or deflation of an inflatable member.

### BACKGROUND

Some implantable medical devices have a pressure sensor to measure the pressure of an inflatable member. However, according to some conventional techniques, it may be difficult to measure (e.g., accurately measure) ambient pressure, which may be used to control inflation and/or deflation of an implantable member by an electronic pump device.

### SUMMARY

In some aspects, the techniques described herein relate to an implantable medical device including: an inflatable member; a fluid reservoir; a pressure sensor connected to the fluid reservoir; and an electronic pump device including a controller configured to execute operations, the operations including: receiving a signal with pressure readings during a time interval from the pressure sensor; identifying a portion of the pressure readings within a percentile range; computing an ambient pressure value based on the portion of the pressure readings; and updating an ambient pressure based on the ambient pressure value.

In some aspects, the techniques described herein relate to a method for measuring ambient pressure in an implantable medical device, the method including: receiving a signal with pressure readings during a time interval from a pressure sensor connected to a fluid reservoir; identifying a portion of the pressure readings within a percentile range; computing an ambient pressure value based on the portion of the pressure readings; and updating an ambient pressure based on the ambient pressure value.

In some aspects, the techniques described herein relate to an implantable medical device including: an inflatable member; a fluid reservoir; a pressure sensor connected to the fluid reservoir; and an electronic pump device including a controller configured to execute operations, the operations including: receiving a signal with pressure readings during a time interval from the pressure sensor; identifying a portion of the pressure readings within a percentile range; computing an ambient pressure value based on the portion of the pressure readings; and updating an ambient pressure based on the ambient pressure value.

In some aspects, the techniques described herein relate to a method for measuring ambient pressure in an implantable medical device, the method including: receiving a signal with pressure readings during a time interval from a pressure sensor connected to a fluid reservoir; identifying a portion of the pressure readings within a percentile range; computing an ambient pressure value based on the portion of the pressure readings; and updating an ambient pressure based on the ambient pressure value.

In some aspects, the techniques described herein relate to a non-transitory computer-readable medium storing executable instructions that cause at least one processor to execute operations, the operations including: receiving a signal with pressure readings during a time interval from a pressure sensor connected to a fluid reservoir, the fluid reservoir configured to be implanted in a body of a patient; identifying a portion of the pressure readings within a percentile range; computing an ambient pressure value based on the portion of the pressure readings; and updating an ambient pressure based on the ambient pressure value.

### BRIEF DESCRIPTION OF THE DRAWINGS

FIG. 1A illustrates an implantable medical device with an electronic pump device for computing an ambient pressure using a pressure sensor connected to a fluid reservoir according to an aspect.

FIG. 1B illustrates an example of a controller for computing an ambient pressure according to an aspect.

FIG. 1C illustrates a histogram of a plurality of pressure readings collected by a pressure sensor according to an aspect.

FIG. 1D illustrates a histogram of a portion of the plurality of pressure readings that fall in a certain percentile range according to an aspect.

FIG. 1E illustrates pressure readings across a plurality of time intervals according to an aspect.

FIG. 2A illustrates an example of a controller that uses a shorter sampling period to determine whether to activate a longer sampling period for computing an ambient pressure value according to an aspect.

FIG. 2B illustrates an example of an exploded view of an electronic pump device according to an aspect.

FIG. 3 illustrates an example of a controller that retrieves a correction value that is used to adjust an ambient pressure according to an aspect.

FIG. 4 illustrates an example of a controller that generates a correction value during a calibration process according to an aspect.

FIG. 5 illustrates an example of a controller with a decimator for downsampling a signal generated by a pressure sensor according to an aspect.

FIG. 6 illustrates a perspective of an inflatable penile prosthesis according to an aspect.

FIG. 7 illustrates an example of an artificial urinary sphincter device according to an aspect.

FIG. 8 illustrates a flowchart depicting example operations of computing the ambient pressure value according to an aspect.

FIG. 9 illustrates an implantable medical device according to another aspect.

### DETAILED DESCRIPTION

This disclosure relates to an implantable medical device configured to detect ambient pressure inside of a body of a patient using reservoir pressure. The ambient pressure may be used to control inflation and/or deflation of an inflatable member. The ambient pressure may be the pressure of the tissues and/or fluids that surround the inflatable medical device, which may be influenced by body position, fluid levels, and/or muscle activity. The implantable medical device includes an inflatable member, a fluid reservoir, and an electronic pump device that transfers fluid between the fluid reservoir and the inflatable member. In some examples, the implantable medical device includes an inflatable penile prosthesis with one or more inflatable cylinders. In some examples, the implantable medical device includes a urinary control device with an inflatable cuff. The electronic pump device may automatically transfer fluid between the inflatable member and the fluid reservoir.

The implantable medical device includes a first pressure sensor connected to the fluid reservoir and a second pressure sensor connected to the inflatable member. The first pressure sensor may detect pressure (e.g., reservoir pressure) in the fluid reservoir. The second pressure sensor may detect pressure (e.g., inflation pressure) in the inflatable member. The electronic pump device includes a controller configured to detect ambient pressure using the first pressure sensor and detect inflation pressure using the second pressure sensor. The controller may compute a gauge pressure using the ambient pressure and the inflation pressure, where the electronic pump device can control inflation and/or deflation of the inflatable member using the gauge pressure.

The controller is configured to determine and update the ambient pressure based on a signal with pressure readings generated by the first pressure sensor. The pressure readings may indicate a pressure level over a time interval. For example, the controller may activate the first pressure sensor to obtain a signal with pressure readings during a time interval (e.g., a set or predetermined period of time) (e.g., a sampling period). The time interval may be thirty seconds, one minute, two minutes, or three minutes, or generally any set length of time. The first pressure sensor may generate the signal with the pressure readings according to a sampling rate. Each pressure reading includes a pressure value, and, in some examples, a timestamp. The controller activates the first pressure sensor to obtain the signal with the pressure readings during the time interval in response to the detection of a triggering event. In some examples, the triggering event may be expiration of a timer or a specified time in a sampling schedule (e.g., one or more times a day, one or more times a week, etc.).

The controller processes the signal to identify a portion of the pressure readings that are within a certain percentile range of the pressure readings during the time interval. The percentile range is defined by a first percentile threshold (e.g., a low percentile threshold) and a second percentile threshold (e.g., a high percentile threshold). In some examples, the percentile range includes or is less than the 50^{th} percentile of the pressure readings. In some examples, the percentile range is the 5^{th} to 50^{th} percentile. In some examples, the percentile range is the 5^{th} to 40^{th} percentile. In some examples, the percentile range is the 5^{th} to 20^{th} percentile. In some examples, the controller uses a lower percentile range to filter out pressure readings caused by short-term drops in pressure and bias due to raised IAP. In some examples, the controller ranks the pressure readings from the signal by the magnitude of the pressure values and selects the pressure readings within the specified percentile range.

The controller computes an ambient pressure value for the time interval based on the portion of the pressure readings from the signal that are within the percentile range. In some examples, the controller selects a pressure value from one of the pressure readings from the signal that are within the percentile range. In some examples, the controller selects a pressure reading with a minimum pressure value among the portion of the pressure readings that are within the percentile range. In some examples, the controller computes an average pressure value from the portion of the pressure readings that are within the percentile range.

In some examples, the controller activates the first pressure sensor to generate a signal with pressure readings according to a first sampling rate (e.g., a higher sampling rate). In some examples, the controller includes a decimator configured to generate a downsampled signal from the signal, where the downsampled signal includes pressure readings according to a second sampling rate (e.g., a lower sampling rate). A decimator may be a digital signal processing (DSP) filter configured to reduce the sampling rate of a discrete-time signal. Then, the controller may identify a portion of the pressure readings from the downsampled signal that are within a certain percentile range of the pressure readings and use those pressure readings to compute an ambient pressure value (e.g., select a pressure reading with a lowest pressure value among the portion that fall within the percentile range).

In some examples, the controller includes logic for determining whether to accept or reject an ambient pressure value computed by the controller for a current time interval. For example, the controller may compute a threshold deviation from a median using the portion of the pressure readings (e.g., the pressure readings that fall within the percentile range). In some examples, the threshold deviation is an absolute deviation. In some examples, the threshold deviation is a standard deviation. In some examples, the threshold deviation is a variance (e.g., averaged squared deviation from the mean). In some examples, the threshold deviation is peak width. The controller may determine whether a deviation of the ambient pressure value from the median is equal to or greater than the threshold deviation. In response to the deviation of the ambient pressure value from the median being equal to or greater than the threshold deviation, the controller may discard (e.g., reject) the ambient pressure value. If rejected, the controller may wait until the next sampling period (e.g., in response to detection of a subsequent triggering event) to re-collect pressure readings to re-compute the ambient pressure value. If rejected, the ambient pressure is not updated (e.g., the controller may continue to use the previous ambient pressure value from a previous time interval for computation of a gauge pressure). In response to the deviation of the ambient pressure value from the median being less than the threshold deviation, the controller may accept the ambient pressure value.

In some examples, the controller may use frequent shorter measurements to detect ambient pressure changes, and, if a large ambient pressure change is detected, the controller may trigger a longer measurement. In some examples, the use of frequent shorter measurements may save the battery life of the pump's battery. The controller periodically initiates the first pressure sensor to generate pressure readings (e.g., first pressure readings) in a first time interval (e.g., a shorter time interval), and, if there is a relatively large difference between the ambient pressure value and a previous ambient pressure value, the controller may activate (e.g., immediately activate) the first pressure sensor to generate pressure readings (e.g., second pressure readings) in a second time interval (e.g., a longer time interval).

For example, the controller may activate the first pressure sensor to generate first pressure readings during a shorter time interval. In response to a difference between an ambient pressure value for the current (e.g., shorter) time interval and an ambient pressure value for a previous time interval being equal to or greater than a threshold level, the controller may activate (e.g., immediately activate) the first pressure sensor to generate second pressure readings in a longer time interval. In response to a difference between an ambient pressure value for the current (e.g., shorter) time interval and an ambient pressure value for a previous time interval being less than a threshold level, the controller may wait for the next measurement period (e.g., the next triggering event). The subsequent triggering event may be the activation of another shorter sampling period after a period of time, or the activation of a longer sampling period.

In some examples, the controller obtains a correction value from a memory device of the electronic pump device and uses the correction value to adjust the ambient pressure. The correction value may be a correction factor that adjusts the ambient pressure to account for the user's body. For example, a bias may exist between the fluid reservoir and ambient pressure that may be unique to the patient, which may depend on the implant location and body composition. In some examples, the correction value is a user-specific value (or user class-specific value). In some examples, the controller may compute the correction value during a calibration process of the implantable medical device. For example, the electronic pump device may include an accelerator. During a calibration process, the user may be instructed to position their body in a plurality of positions, where the controller receives acceleration data from the accelerator. The controller may generate the correction value based on the acceleration data. The use of the accelerator may assist with determining the height differential between the fluid reservoir and the inflatable member and/or assist with correcting the changes to the reservoir pressure caused by position change and movement.

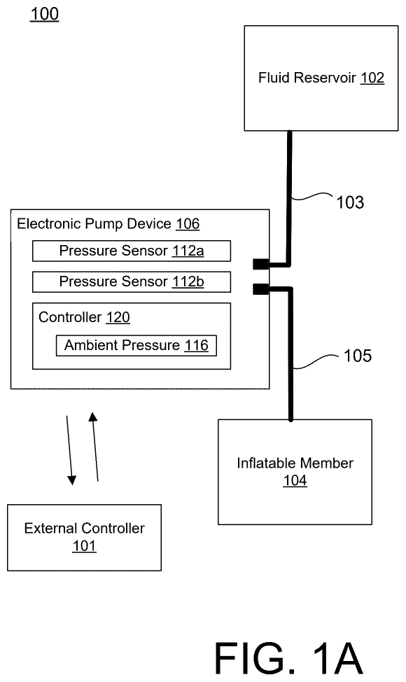

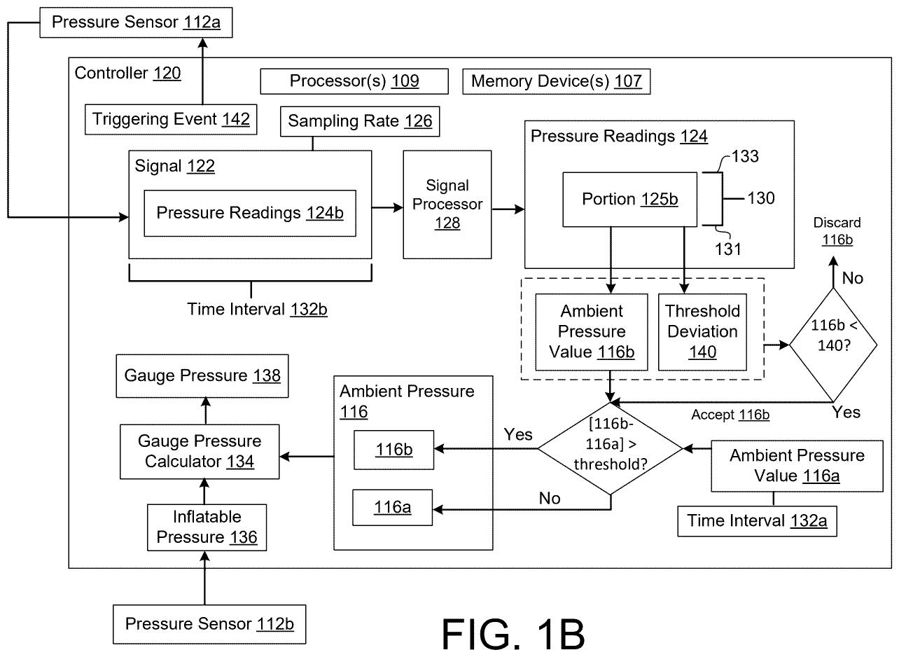

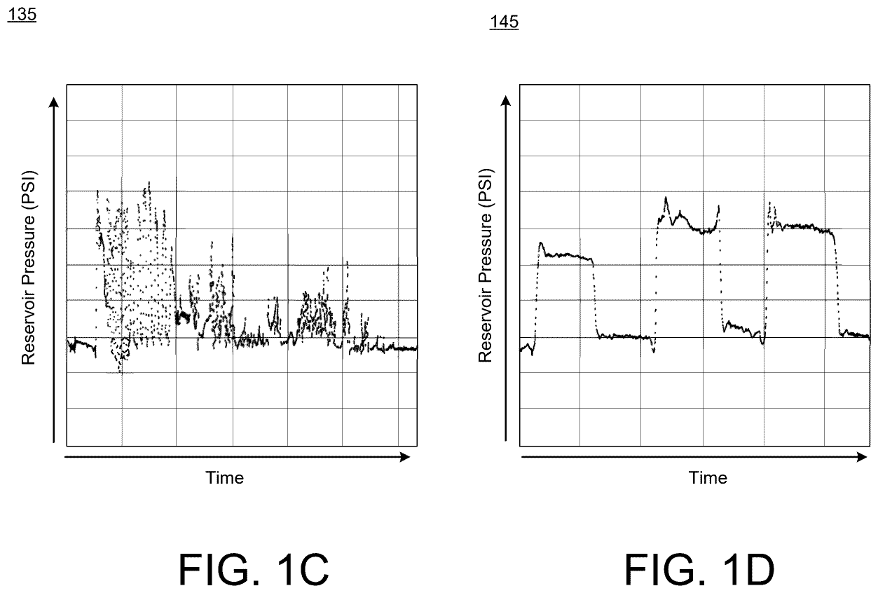

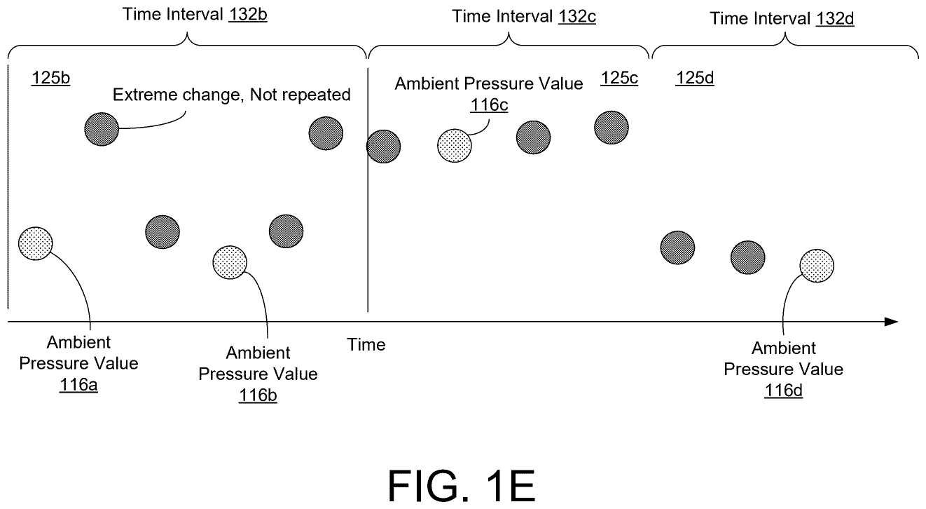

FIGS. 1A to 1E illustrate an implantable medical device 100 that monitors and computes ambient pressure 116 inside of a body of a patient using a pressure sensor 112a connected to a fluid reservoir 102. In some examples, the implantable medical device 100 is an artificial urinary sphincter device. In some examples, the implantable medical device 100 is an inflatable penile prosthesis. However, the implantable medical device 100 may include any type of medical device that transfers fluid between components of the implantable medical device 100.

As shown in FIG. 1A, the implantable medical device 100 includes a fluid reservoir 102, an inflatable member 104, and an electronic pump device 106 configured to transfer fluid between the fluid reservoir 102 and the inflatable member 104. In some examples, the inflatable member 104 is an inflatable cuff member configured to be implemented around a urethra of a patient. In some examples, the inflatable member 104 is a penile prosthetic with one or more inflatable cylinders that may be implanted into the corpus cavernosum of the user. The fluid reservoir 102 may be implanted in the abdomen or pelvic cavity of the user (e.g., the fluid reservoir 102 may be implanted in the lower portion of the user's abdominal cavity or the upper portion of the user's pelvic cavity). In some examples, at least a portion of the electronic pump device 106 may be implemented in the patient's body.

The inflatable member 104 may be capable of expanding upon the injection of fluid into a cavity of the inflatable member 104. If implanted around the urethra, the expansion of the inflatable member 104 causes the urethra to become restricted, thereby reducing the risk of incontinence in patients. For example, the electronic pump device 106 is configured to move fluid to pressure the inflatable cuff (e.g., the inflatable member 104), which constricts the urethra, thereby restricting the flow of urine. To urinate, the patient may operate the electronic pump device 106 to depressurize the inflatable cuff by transferring fluid from the inflatable cuff to the fluid reservoir 102. If implanted into the corpus cavernosum, upon injection of the fluid into the inflatable member 104, the inflatable member 104 may increase its length and/or width, as well as increase its rigidity.

The fluid reservoir 102 may include a container having an internal chamber configured to hold or house fluid that is used to inflate the inflatable member 104. In some examples, the fluid reservoir 102 is pressurized. In some examples, the fluid reservoir 102 is a pressurized balloon. In some examples, the implantable medical device 100 includes a single pressurized balloon. In some examples, the implantable medical device 100 includes two or more pressurized balloons. The pressure in the inflatable member 104 may be generated by the fluid reservoir 102. In some examples, the pressure in the fluid reservoir 102 is greater than the pressure in the inflatable member 104 (e.g., even when the inflatable member 104 is at its target or maximum pressure). In some examples, the pressure in the fluid reservoir 102 is always greater than the pressure in the inflatable member 104.

The implantable medical device 100 may include a first tube member 103 and a second tube member 105. In some examples, the first tube member 103 and the second tube member 105 are referred to as conduit connectors. Each of the first tube member 103 and the second tube member 105 may define a lumen configured to transfer the fluid to and from the electronic pump device 106. The first tube member 103 may be coupled to the electronic pump device 106 and the fluid reservoir 102 such that fluid can be transferred between the electronic pump device 106 and the fluid reservoir 102 via the first tube member 103. For example, the first tube member 103 may define a first lumen configured to transfer fluid between the electronic pump device 106 and the fluid reservoir 102. The first tube member 103 may include a single or multiple tube members for transferring the fluid between the electronic pump device 106 and the fluid reservoir 102. In some examples, the first tube member 103 may be referred to as first tube members, and two first tube members can be connected together using a connector.

The second tube member 105 may be coupled to the electronic pump device 106 and the inflatable member 104 such that fluid can be transferred between the electronic pump device 106 and the inflatable member 104 via the second tube member 105. For example, the second tube member 105 may define a second lumen configured to transfer fluid between the electronic pump device 106 and the inflatable member 104. The second tube member 105 may include a single or multiple tube members for transferring the fluid between the electronic pump device 106 and the inflatable member 104. In some examples, the second tube member 105 may be referred to as second tube members, and two second tube members can be connected together using a connector. In some examples, the first tube member 103 and the second tube member 105 may include a silicone rubber material. In some examples, the electronic pump device 106 may be directly connected to the fluid reservoir 102.

The electronic pump device 106 that can monitor control and regulate the pressure within an inflatable member 104. In some examples, the electronic pump device 106 is referred to as a can. The electronic pump device 106 may automatically transfer fluid between the fluid reservoir 102 and the inflatable member 104 without the user manually operating a pump (e.g., squeezing and releasing a pump bulb). The electronic pump device 106 includes pumps, valves, a battery, and electronic circuitry. The electronic pump device 106 may include an antenna configured to wirelessly transmit (and receive) wireless signals from an external device 101. The external device 101 may be any type of component that can communicate with the electronic pump device 106. The external device 101 may be a computer, smartphone, tablet, pendant, key fob, etc. A user may use the external device 101 to control the implantable medical device 100. In some examples, the user may use the external device 101 to inflate or deflate the inflatable member 104.

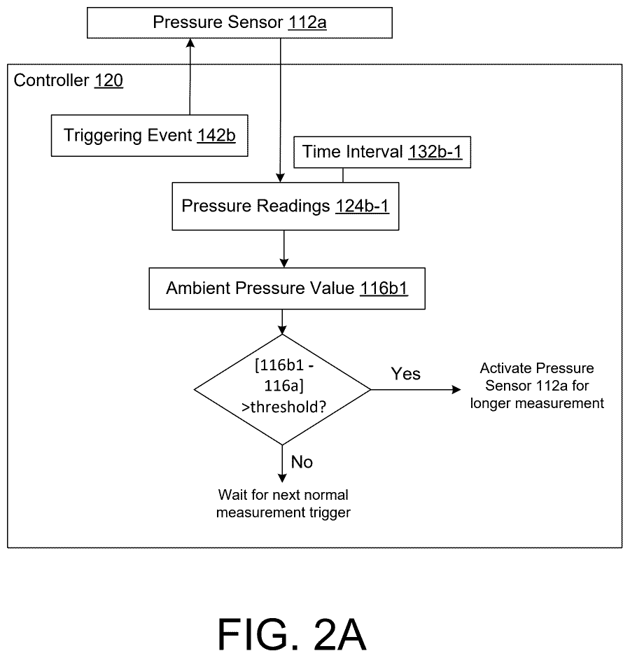

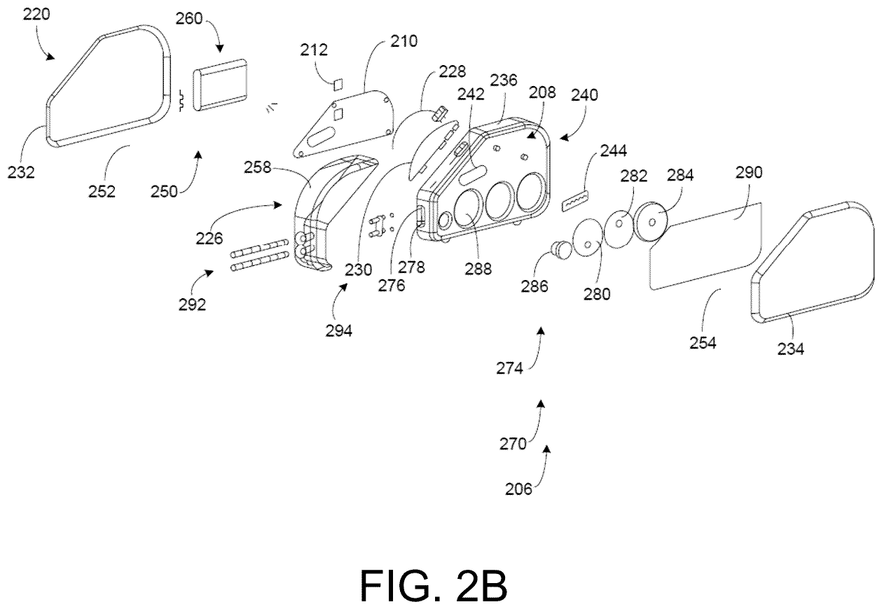

The electronic pump device 106 includes one or more pressure sensors. The electronic pump device 106 may include a pressure sensor 112a connected to the fluid reservoir 102. In some examples, the pressure sensor 112a is coupled to a fluid port (e.g., first port 276 or second port 278 of FIG. 2B) inside of a housing of the electronic pump device 106, where the fluid port is in fluid communication with the fluid reservoir 102. In some examples, the pressure sensor 112a is aligned (e.g., in-line) with the fluid port. In some examples, the pressure sensor 112a is coupled to a circuit substrate (e.g., the circuit substrate 210 of FIG. 2B). The pressure sensor 112a is used for measuring a pressure (e.g., reservoir pressure) of the fluid reservoir 102. The electronic pump device 106 includes a pressure sensor 112b connected to the inflatable member 104. The pressure sensor 112b is used for measuring an inflatable pressure 136 (e.g., inflation pressure) of the inflatable member 104. In some examples, the pressure sensor 112b is coupled to a fluid port (e.g., first port 276 or second port 278 of FIG. 2B) inside of a housing of the electronic pump device 106, where the fluid port is in fluid communication with the inflatable member 104. In some examples, the pressure sensor 112b is aligned (e.g., in-line) with the fluid port fluidly connected to the inflatable member 104. In some examples, the pressure sensor 112b is coupled to a circuit substrate (e.g., the circuit substrate 210 of FIG. 2B).

The electronic pump device 106 includes a controller 120 configured to monitor, compute, and/or update an ambient pressure 116. The ambient pressure 116 may be used to control inflation and/or deflation of an inflatable member 104. The ambient pressure 116 may be the pressure of the tissues and/or fluids that surround the implantable medical device 100, which may be influenced by body position, fluid levels, and/or muscle activity. In some examples, the ambient pressure 116 may be dependent upon altitude, temperature, humidity, and/or other weather conditions. In some examples, the ambient pressure 116 is referred to as atmospheric pressure. In some examples, the ambient pressure 116 is referred to as a local atmospheric pressure.

In some examples, the controller 120 determines the ambient pressure 116 using the pressure sensor 112a (e.g., a pressure sensor connected to the fluid reservoir 102). In some examples, cylinder pressure sensors (e.g., pressure sensor 112b) may be biased above or below ambient pressure 116 by movement, and/or manipulation, etc. However, in some examples, bias on the reservoir sensor may be caused by IAP changes and/or external pressures, which typically increases pressure (e.g., only increases). By using a pressure sensor connected to the fluid reservoir 102 for computing ambient pressure 116, the implantable medical device 100 may be less susceptible to being affected (e.g., significantly affected) by temporary or minor errors or inconsistencies in measurements (e.g., it can handle short-term fluctuations or inaccuracies in data without being significantly impacted).

Referring to FIG. 1B, the controller 120 includes one or more processors 109 and one or more memory devices 107. A memory device 107 may store an ambient pressure 116 that is used to compute a gauge pressure 138, and the controller 120 may periodically update the ambient pressure 116 and store the updated ambient pressure 116 in the memory device 107.

The controller 120 may activate the pressure sensor 112a to generate a signal 122 with pressure readings 124b during a time interval 132b. The time interval 132b may have a predetermined length (e.g., thirty seconds, one minute, two minutes, or three minutes, or generally any set length of time). In some examples, a time interval is referred to as a sampling period or a measurement period. In some examples, the controller 120 may periodically activate the pressure sensor 112a to generate the signal 122 for a respective sampling period and determine whether to update the ambient pressure 116. In some examples, updating the ambient pressure 116 includes replacing an old value with a new value in the memory device 107.

The controller 120 activates the pressure sensor 112a to generate the signal 122 with the pressure readings 124b during the time interval 132b in response to the detection of a triggering event 142. In some examples, the triggering event 142 may be expiration of a timer or achieving a time indicated by a sampling schedule (e.g., one or more times a day, one or more times a week, etc.). The pressure sensor 112a may generate the signal 122 with pressure readings 124b according to a sampling rate 126. In some examples, the sampling rate 126 is between 1-10 Hz. Each pressure reading 124b includes a pressure value, and, in some examples, a timestamp. FIG. 1C illustrates a histogram 135 of a plurality of pressure readings 124b collected by a pressure sensor 112a according to an aspect.

The controller 120 includes a signal processor 128 that processes the signal 122 to identify a portion 125b of the pressure readings 124b that are within a certain percentile range 130 of the pressure readings 124b during the time interval 132b. The percentile range 130 is defined by a percentile threshold 131 (e.g., a low percentile threshold) and a percentile threshold 133 (e.g., a high percentile threshold). In some examples, the percentile range includes or is less than 50^{th} percentile of the pressure readings. In some examples, the percentile range is 5^{th} to 50^{th} percentile. In some examples, the percentile range is 5^{th} to 40^{th} percentile. In some examples, the percentile range is 5^{th} to 20^{th} percentile. In some examples, the controller uses a lower percentile range to filter out pressure readings 124b caused by short-term drops in pressure and bias due to raised IAP. In some examples, the controller 120 ranks the pressure readings 124b from the signal 122 by the magnitude of the pressure values and selects the pressure readings 124b within the specified percentile range. FIG. 1D illustrates a histogram 145 of a portion 125b of the plurality of pressure readings 124b that fall in a certain percentile range 130 according to an aspect.

The controller 120 generates an ambient pressure value 116b based on the portion 125b of the pressure readings 124b from the signal 122 that are within the percentile range 130. In some examples, the controller 120 selects a pressure value from one of the pressure readings 124b from the signal 122 that are within the percentile range 130. In some examples, the controller 120 selects a pressure reading 124b with a minimum pressure value among the portion 125b of the pressure readings 124b that are within the percentile range 130. In some examples, the controller 120 computes an average pressure value from the portion 125b of the pressure readings 124 that are within the percentile range 130.

In some examples, the controller 120 includes logic for determining whether to accept or reject the ambient pressure value 116b computed by the controller 120 for a current time interval (e.g., time interval 132b). For example, the controller 120 may compute a threshold deviation 140 using the portion 125b of the pressure readings 124b (e.g., the pressure readings 124b that fall within the percentile range 130).

In some examples, the threshold deviation 140 is an absolute deviation. In some examples, the threshold deviation 140 is a standard deviation. In some examples, the threshold deviation 140 is a variance (e.g., averaged squared deviation from the mean). In some examples, the threshold deviation 140 is peak width.

The controller 120 may determine whether a deviation of the ambient pressure value 116b from the median is equal to or greater than the threshold deviation 140. In response to the deviation of the ambient pressure value 116b from the median being equal to or greater than the threshold deviation 140, the controller 120 may discard (e.g., reject) the ambient pressure value 116b. If rejected, the controller 120 may wait until the next sampling period (e.g., in response to detection of a subsequent triggering event 142) to re-collect pressure readings (e.g., time interval 132c) to re-compute the ambient pressure value (e.g., ambient pressure value 116c). In response to the deviation of the ambient pressure value 116b from the median being less than the threshold deviation 140, the controller 120 may accept the ambient pressure value 116b.

In some examples, when the ambient pressure value 116b is accepted, the controller 120 may compare the ambient pressure value 116b for the current time interval (e.g., time interval 132b) with an ambient pressure value 116a from a previous time interval 132a to determine whether to update the ambient pressure 116 with the ambient pressure value 116b (e.g., a new ambient pressure value) for the current time interval (e.g., time interval 132b) or continue to maintain the previous ambient pressure value 116a. In some examples, if the difference between the ambient pressure value 116b for the current time interval 132b and the ambient pressure value 116a for the previous time interval 132a is equal to or greater than a threshold level, the controller 120 may update the ambient pressure 116 with the ambient pressure value 116b for the current time interval 132b.

If the difference between the ambient pressure value 116a for the current time interval 132b and the ambient pressure value 116a for the previous time interval 132a is less than the threshold level, the controller 120 may use the previous ambient pressure value 116a (e.g., the ambient pressure 116 is not updated). In some examples, if the ambient pressure value 116b for the current time interval 132b is less than the ambient pressure value 116a for the previous time interval 132a, the controller 120 uses the new ambient pressure value (e.g., the ambient pressure value 116b). In some examples, if the ambient pressure value 116b for the current time interval 132b is greater than the previous ambient pressure value 116a by a threshold level, the controller 120 uses the new ambient pressure value (e.g., the ambient pressure value 116b).

The controller 120 detects an inflatable pressure 136 of the inflatable member 104 using the pressure sensor 112b. The controller 120 includes a gauge pressure calculator 134 computes a gauge pressure 138 using the ambient pressure 116 and the inflatable pressure 136. In some examples, the gauge pressure calculator 134 offsets the inflatable pressure 136 by the ambient pressure 116.

The memory device(s) 107 may store executable instructions that when executed by the processor(s) 109 that cause the processor(s) to execute the operations of the controller 120 as discussed herein. In some examples, the memory device(s) 107 include a non-transitory computer-readable medium or computer program product. In some examples, the controller 120 and the pressure sensors (e.g., pressure sensor 112a, pressure sensor 112b) may be stored on a printed circuit board in a housing of the electronic pump device 106. In some examples, the controller 120 is included in a printed circuit board and is attached to a manifold structure that also includes one or more pumps and one or more valves for transferring fluid between the inflatable member 104 and the fluid reservoir 102.

FIG. 1E illustrates pressure readings for three time intervals, e.g., time interval 132b, time interval 132c, and time interval 132d. The time intervals may not be directly adjacent to each other, where a period of time exists between successive time intervals. In some examples, the length of the time interval 132b, the time interval 132c, and the time interval 132d is the same. In the time interval 132b, a portion 125b of pressure readings are selected that achieve a percentile range 130. The controller 120 determines that an ambient pressure value 116b is the lowest among the portion 125b of the pressure readings in the time interval 132b and updates the ambient pressure 116 with the ambient pressure value 116b. In some examples, the previous ambient pressure value is an ambient pressure value 116a, and, since the ambient pressure value 116b is less than the previous ambient pressure value, the controller 120 updates the ambient pressure 116 in the memory device 107 from the ambient pressure value 116a to the ambient pressure value 116b.

In a subsequent time interval (e.g., time interval 132c), a portion 125c of pressure readings are selected that achieve the percentile range 130. The controller 120 determines that an ambient pressure value 116c is the lowest among the portion 125c of the pressure readings in the time interval 132c and updates the ambient pressure 116 with the ambient pressure value 116c in the memory device 107. In the time interval 132c, a large increase in pressure values is repeatedly observed, and, therefore, the previous ambient value (e.g., ambient pressure value 116b) is discarded, and the lowest pressure value (e.g., the ambient pressure value 116c) in the time interval 132c is selected as the new pressure value for the ambient pressure 116.

In another subsequent time interval (e.g., time interval 132d), a portion 125d of pressure readings are selected that achieve the percentile range 130. The controller 120 determines that an ambient pressure value 116d is the lowest among the portion 125d of the pressure readings in the time interval 132d and updates the ambient pressure 116 with the ambient pressure value 116d in the memory device 107. In the time interval 132d, a decrease in pressure values is repeatedly observed, and, therefore, the previous ambient value (e.g., ambient pressure value 116c) is discarded, and the lowest pressure value (e.g., the ambient pressure value 116d) in the time interval 132d is selected as the new pressure value for the ambient pressure 116.

FIG. 2A illustrates an example of a controller 120 that uses a shorter sampling period to determine whether to activate a longer sampling period for computing an ambient pressure value according to an aspect. Referring to FIG. 2A, the controller 120 may use frequent shorter measurements to detect ambient pressure changes, and, if a large ambient pressure change is detected, the controller 120 may trigger a longer measurement. The use of frequent shorter measurements may increase the amount of time that a pump's battery can operate before the battery may be required to be recharged.

The controller 120 initiates (e.g., periodically initiates) the pressure sensor 112a to generate pressure readings 124b-1 (e.g., first pressure readings) in a time interval 132b-1. The time interval 132b-1 may have a length that is shorter than the normal time interval (e.g., time interval 132b). In some examples, the time interval 132b-1 may be referred to as a shorter time interval. The controller 120 may compute an ambient pressure value 116b1 using the pressure readings 124b-1. In some examples, the controller 120 may select a pressure reading 124b-1 with a lowest value as the ambient pressure value 116b1.

If there is a relatively large difference between the ambient pressure value 116b1 and a previous ambient pressure value 116a, the controller 120 may activate (e.g., immediately activate) the pressure sensor 112a to generate pressure readings (e.g., pressure readings 124b) (e.g., second pressure readings) in the time interval 132b. In some examples, the time interval 132b has a length that is longer than the time interval 132b-1. For example, if the difference between the ambient pressure value 116b1 and the previous ambient pressure value 116a (e.g., computed in a previous (longer) time interval 132a) is equal to or greater than a threshold level, the controller 120 activates (e.g., immediately activates) the pressure sensor 112a to collect pressure readings 124b during a longer interval (e.g., the time interval 132b).

In response to a difference between the ambient pressure value 116b1 for the current (e.g., shorter) time interval 132b-1 and the ambient pressure value 116a for a previous time interval 132a being detected as less than a threshold level, the controller 120 may wait for the next sampling period (e.g., the next triggering event 142 or 142b). The subsequent triggering event may be the activation of another shorter sampling period (e.g., time interval 132b-1), or the activation of a longer sampling period (e.g., time interval 132b).

FIG. 2B illustrates an example of an exploded view of an electronic pump device 206 according to an aspect. The electronic pump device 206 may be an example of the electronic pump device 106 and may include any of the details discussed with reference to the other figures. The electronic pump device 206 includes a housing 220 with a first sidewall 232, a second sidewall 234, a peripheral wall 236, and a frame 240. The first sidewall 232, second sidewall 234, and the peripheral wall 236 are hermetically sealed together to form an internal compartment 250 within the housing 220.

The frame 240 is disposed within the internal compartment 250 to form a first partition 252 and a second partition 254 in such a manner that the first partition 252 is hermetically sealed from the second partition 254. The frame 240 can be integrally formed with the peripheral wall 236, the first sidewall 232, and/or the second sidewall 234. In some examples, the frame 240 is welded to the peripheral wall 236 or welded to the first sidewall 232, and/or the second sidewall 234. The first sidewall 232, the peripheral wall 236, and the frame 240 may form the first partition 252. The second sidewall 234, the peripheral wall 236, and the frame 240 may form the second partition 254, which is opposite the frame 240 from the first partition 252.

The electronic pump device 206 can include a header 226 attached to the housing 220 to form an internal region 258 between an inner surface of the header 226 and an outer surface of the housing 220 that includes power and communication interface structures such as a secondary coil 228 and the antenna 230 external to the hermetically sealed housing 220. The header 226 is configured from a dielectric or insulative material, such as a radome, to allow the transmission of power and communication signals between the antenna 230 and a handset programmer or charger, and between the secondary coil 228 and the charger. For example, the header 226 may include an over molded polymer affixed to the housing 220 and including the secondary coil 228 and the antenna 230 within the internal region 258. The secondary coil 228 and antenna 230 are constructed from a biocompatible material. In some examples, the secondary coil 228 and antenna 230 can be formed as a coil from a stamped titanium core clad with gold or silver. In some examples, the secondary coil 228 and antenna 230 can be formed from a gold wire.

The electronic pump device 206 includes an energy storage system, such as a battery (e.g., a rechargeable power source) (e.g., a rechargeable battery), and electronic components 212 within the first partition 252. The electronic components 212 can be disposed on a circuit substrate 210, such as a plurality of circuit boards, within the first partition 252. The battery 260 can assume various forms appropriate to provide power for generating desired electrical signals and to store power provided from the electronic components 212. For example, the battery 260 can incorporate lithium-ion (Li+) chemistry, e.g., a lithium-ion battery to operate the electronic components 212. In some examples, the electronic components 212 can be implemented by various components including resistors, capacitors, transistors, and integrated circuits disposed on the circuit substrate 210. The secondary coil 228 and antenna 230 are electrically coupled to the electronic components 212 within the first partition 252, such as via a hermetic feedthrough component.

The electronic components 212 can include a recharge system, a communication system, and a controller. The recharge system includes hardware configured to interface with the secondary coil 228 to receive power signals, and to provide the power signals in a form suitable to recharge the battery 260 and can include circuitry to reduce the likelihood of overcharging the battery 260. The communication system includes hardware configured to interface with the antenna 230 to receive electrical communication signals. For instance, the communication system can be configured to communicate via a wireless personal area network technology such as a short-range communication protocol (e.g., Bluetooth) (e.g., Bluetooth Low Energy), which is compatible with several operating systems that can be applied in mobile devices configured as external devices (e.g., handset programmers). The communication system can include an integrated circuit to implement an applied communication technology. In some examples, the communication system can be used to transmit communication signals to other devices, such as a charger or the handheld programmer (e.g., external device), and the communication system can be implemented to generate communication signals and provide the communication signals to the antenna 230 for transmission. In some examples, the communication system can be configured to receive and transmit radio frequency signals via the antenna 230. The controller can include a microcontroller to operate the recharge system and to receive and operate in response to communication signals or generate communication signals from the communication system.

The electronic pump device 206 also includes a fluidic circuit 270 within the second partition 254 and opposite the frame 240 from the battery 260 and electronic components 212. In some examples, the frame 240 can include an opening 242 that includes a hermetic interface 244, such as a feedthrough hermetically affixed to the frame 240. The electronic components 212 are operably coupled to the fluidic circuit 270 across the frame 240 via the hermetic interface 244. For example, the controller of the electronic components 212, powered by the battery 260, can cause the operation of the fluidic circuit 270 such as to control and monitor the fluidic circuit 270.

The fluidic circuit 270 includes a fluidic manifold 208 and fluidic components 274 operably coupled to the fluidic manifold 208. In some examples, the fluidic manifold 208 is a structure integrated into the frame 240 such that the fluidic manifold 208 and the frame 240 together form the hermetic barrier between the first partition 252 and the second partition 254 of the internal compartment 250. For instance, the battery 260, the circuit substrate 210, or electronic components 212 can be coupled to a first major surface of the fluidic manifold 208 in the first partition 252, and the fluidic components 274 are operably coupled to a second, and opposite major surface of the fluidic manifold 208 in the second partition 254.

The fluidic circuit 270 provides for the transfer of the fluid between the fluid reservoir (e.g., the fluid reservoir 102) and the inflatable member (e.g., the inflatable member 104). The fluidic manifold 208, which can be a hermetic manifold, segments and contains the fluid from the internal compartment 250 to reduce the chance of fluid exchange and directs the fluid from a first port 276 to a second port 278 via internal fluid passageways or channels.

The fluidic components 274 include a plurality of fluid pumps, such as pumps 280, 282, a valve 284 mounted into the fluidic manifold 208 in fluidic communication with a manifold passageway to transfer fluid from the first port 276 to the second port 278. The pumps and the valve(s) are in fluid communication with a single fluid passageway between ports 276, 278. The fluidic components 274 also includes one or more pressure sensors 286 operably coupled to the fluidic manifold 208 and in fluidic communication with the passageway to detect a pressure of the fluid within the fluidic manifold 208.

In some examples, the fluidic components 274 are included in a planar configuration on the fluidic manifold 208 in which the pumps 280, 282, valve 284, and pressure sensor 286 are mounted into the fluidic manifold 208 on a plane for slim profile within the second partition 254. The fluidic manifold 208 can include chambers 288 formed into the second major surface in which the chambers are fluidically coupled to the single passageway within the fluidic manifold 208. The chambers are configured to receive the pumps 280, 282, and valve 284 and one or more pressure sensors 286. In some examples, the fluidic manifold 208 can receive a piezoelectric pump. The fluidic manifold 208 can receive a component cover 290 over the fluidic components 274, which can be hermetically sealed to the second major surface.

In some examples, the electronic pump device 206 may include kink resistant tubing 292 that can extend through the header 226 and attach to the ports 276, 278 via components such as a barb 294 and O-rings. The kink resistant tubing 292 is or attached to the tube members (e.g., tube members 103, 105) to fluidically couple the electronic pump device 206 to the fluid reservoir and the inflatable member.

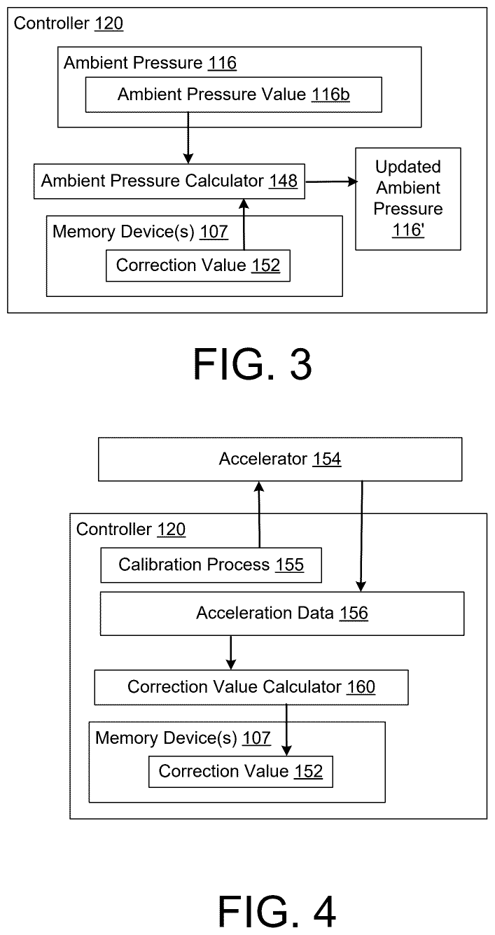

FIG. 3 illustrates an example of a controller 120 that retrieves a correction value 152 that is used to adjust an ambient pressure 116 according to an aspect. Referring to FIG. 3, the controller 120 may obtain a correction value 152 from a memory device 107 of the electronic pump device 106 and use the correction value 152 to adjust the ambient pressure 116.

For example, the controller 120 may include an ambient pressure calculator 148 that retrieves a correction value 152 from the memory device 107. The ambient pressure calculator 148 may also receive the ambient pressure value 116b from the memory device 107 and generate an updated ambient pressure 116′ using the correction value 152. In some examples, the correction value 152 is stored in the memory device 107 before the implantable medical device 100 is implanted in the patient's body. In some examples, the controller 120 stores the correction value 152 after the implantable medical device 100 is implanted in the patient's body. In some examples, the controller 120 receives the correction value 152 from the external device 101. In some examples, the controller 120 generates (e.g., determines) the correction value 152 during a set-up or calibration process of the implantable medical device 100.

The updated ambient pressure 116′ may be an ambient pressure value that accounts for the user's body. For example, the correction value 152 may be a correction factor that adjusts the ambient pressure 116 to account for the user's body. For example, a bias may exist between the fluid reservoir 102 and ambient pressure 116 that may be unique to the patient, which may depend on the implant's location and user's body composition. In some examples, the correction value 152 is a user-specific value (or user class-specific value). In some examples, the gauge pressure calculator 134 may use the updated ambient pressure 116′ to compute the gauge pressure 138.

FIG. 4 illustrates an example of a controller 120 that generates a correction value 152 during a calibration process 155 according to an aspect. In some examples, as shown in FIG. 4, the controller 120 may compute the correction value 152 during a calibration process 155 of the implantable medical device 100. In some examples, the electronic pump device 106 may include an accelerator 154 configured to detect an acceleration of the electronic pump device 106 about an x-axis, y-axis, and z-axis. In some examples, in response to initiation of the calibration process 155, the controller 120 may activate the accelerator 154 to obtain the acceleration data 156. The accelerometer data 156 includes information about the acceleration in an x-axis, a y-axis, and a z-axis.

During the calibration process 155, the user may be instructed to position their body in a plurality of positions, where the controller 120 receives acceleration data 156 about the plurality of positions from the accelerator 154. The controller 120 may include a correction value calculator 160 that generates the correction value 152 based on the acceleration data 156. The correction value calculator 160 stores the correction value 152 in the memory device 107, which is used to adjust the ambient pressure 116. The use of the accelerator 154 may assist with determining the height differential between the fluid reservoir 102 and the inflatable member 104 and/or assist with correcting the changes to the reservoir pressure caused by position change and movement.

In some examples, the accelerometer 154 can be used to further improved the accuracy of the ambient pressure 116 (e.g., the local environmental pressure measurement) by determining the orientation of the patient. The orientation of the patient may have an impact on the ambient pressure 116 and/or the inflatable pressure 136. The accelerometer 154 could also be utilized for motion detection. The degree of activity could be correlated and associated with increases in intra-abdominal pressure which would be transferred to the reservoir 102 and thus impact the associated pressure reading (e.g., pressure reading 124b) on this component. Compensation for these intra-abdominal pressure increases due to activity could further increase the accuracy of the gauge pressure 138 by adjusting the reading obtained by the pressure sensor 112a monitoring the reservoir 102. This could inform the gauge pressure 138 by incorporating the calculated change in resting pressure to the activity pressure in order to determine current ambient pressure 116.

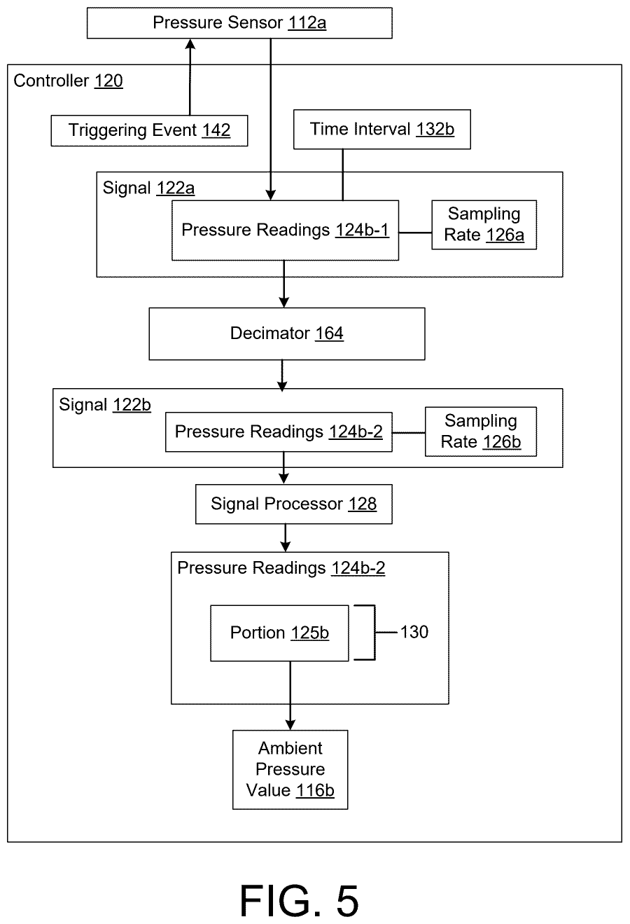

FIG. 5 illustrates an example of a controller 120 with a decimator 164 to downsample a signal 122a generated by a pressure sensor 112a. In response to a triggering event 142, the controller 120 activates the pressure sensor 112a to generate a signal 122a with pressure readings 124b-1 during a time interval 132b according to a sampling rate 126a (e.g., a higher sampling rate). The controller 120 includes a decimator 164 configured to generate a signal 122b (e.g., a downsampled signal) from the signal 122a, where the signal 122b includes pressure readings 124b-2 according to a sampling rate 126b (e.g., a lower sampling rate). The number of pressure readings 124b-2 is less than the number of pressure readings 124b-1. The decimator 164 may be a digital signal processing (DSP) filter used to reduce the sampling rate of a discrete-time signal. Then, the controller 120 may identify a portion 125b of the pressure readings 124b-2 from the signal 122b (e.g., the downsampled signal) that are within a certain percentile range 130 of the pressure readings 124b-2 and may use that portion 125b to generate an ambient pressure value 116b (e.g., select a pressure reading with a lowest pressure value among the portion 125b that fall within the percentile range 130).

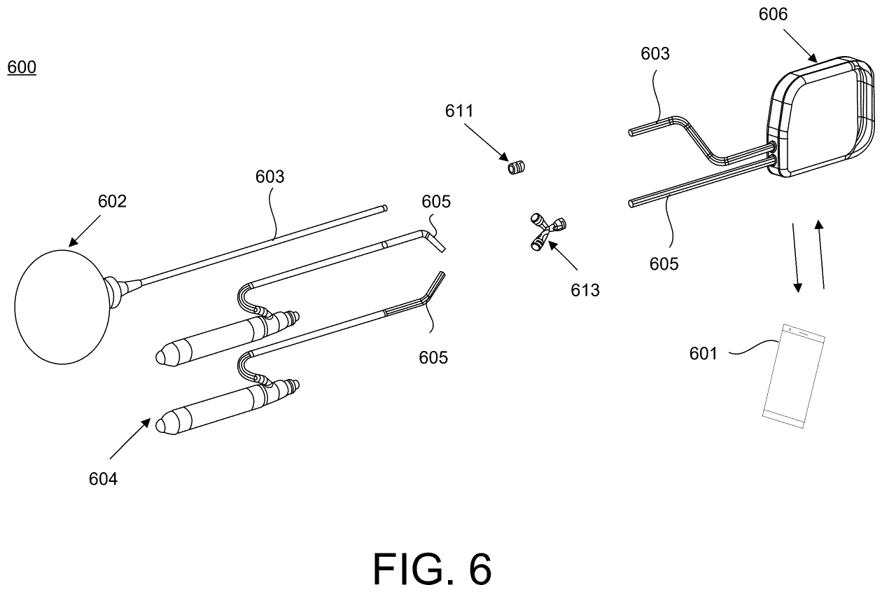

FIG. 6 illustrates a perspective of an inflatable penile prosthesis 600 according to an aspect. The inflatable penile prosthesis 600 may be an example of any of the medical devices discussed herein (e.g., including implantable medical device 100), and, therefore, may include any of the details discussed with reference to the previous figures.

The inflatable penile prosthesis 600 includes an inflatable member 604, a fluid reservoir 602, and an electronic pump device 606. The inflatable member 604 includes a pair of inflatable cylinders. The electronic pump device 606 may be an example of any of the pump devices discussed with reference to the previous figures and may include any of the details discussed herein. The electronic pump device 606 includes fluidics components such as pumps, valves, and/or sensing devices positioned in fluid passageways. The pump device 606 includes components such as, for example, one or more fluid control devices, one or more pressure sensors, and other such components. The electronic pump device 606 includes an electronic control system configured to provide for the transfer of fluid between a reservoir 602 and an inflatable member 604 via the fluidics components.

The electronic pump device 606 may include a controller (e.g., the controller 120) and pressure sensors (e.g., pressure sensor 112a, pressure sensor 112b). In some examples, the controller is included in a printed circuit board that is included in a housing of the electronic pump device 606. Fluidics components and the electronic components of the electronic pump device 106 are included in a housing. In some examples, fluidics components and electronic components in the housing define a manifold (e.g., an electronically controlled fluid manifold) that provides for the electronic control of the flow of fluid between the reservoir 602 and the inflatable member 604. In some examples, the electronic pump device 606 can communicate with an external device 601, via respective communication modules. For example, an application stored in a memory and executed by a processor of the external device 601 may allow the user and/or a physician to operate, view, monitor and alter operation of the inflatable penile prosthesis 600.

The inflatable penile prosthesis 600 includes one or more first tube members 603 that connect a first fluid port of the electronic pump device 606 with the reservoir 602. One or more second tube members 605 connect a second fluid port of the electronic pump device 606 with the inflatable member 604 in the form of the inflatable cylinders. In some examples, the inflatable penile prosthesis 600 includes a connector 611 that is used to connect two tube members 603 together, and a connector 613 that is used to connect two tube members 605 together.

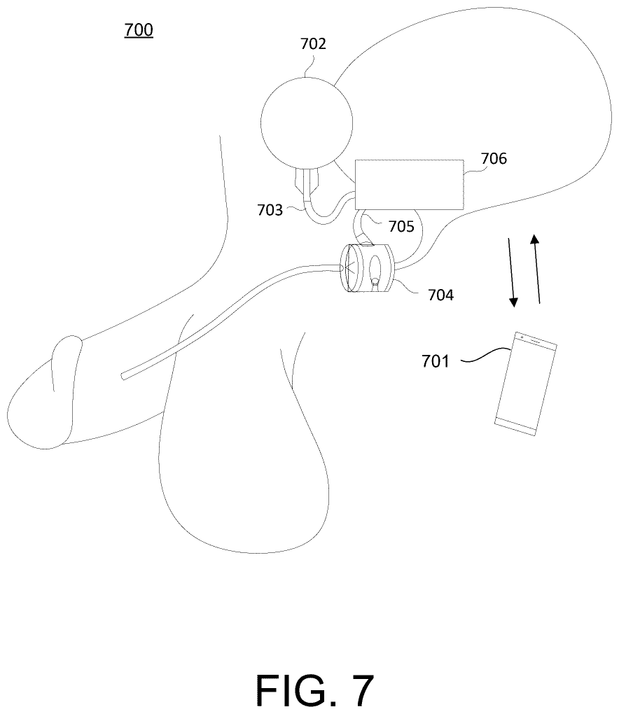

FIG. 7 illustrates a urinary control device 700 having an electronic pump device 706 according to an aspect. The urinary control device 700 may be an example of the implantable medical device 100. In some examples, the urinary control device 700 is an artificial urinary sphincter device. The electronic pump device 706 may include any of the features of the pump devices discussed herein. The urinary control device 700 includes an electronic pump device 706, a fluid reservoir 702, and a cuff 704 (e.g., an inflatable cuff).

The fluid reservoir 702 may be a pressure-regulating inflation balloon or element. The fluid reservoir 702 is in operative fluid communication with the cuff 704 via one or more tube members 703, 705. The fluid reservoir 702 is constructed of polymer material that is capable of elastic deformation to reduce fluid volume within the fluid reservoir 702 and push fluid out of the fluid reservoir 702 and into the cuff 704. However, the material of the fluid reservoir 702 can be biased or include a shape memory construct adapted to generally maintain the fluid reservoir 702 in its expanded state with a relatively constant fluid volume and pressure. In some examples, this constant level of pressure exerted from the fluid reservoir 702 to the cuff 704 will keep the cuff 704 at a desired inflated state when open fluid communication is provided between the fluid reservoir 702 and the cuff 704. In some examples, the fluid reservoir 702 is implanted into the abdominal space.

A user may use an external device 701 to control the urinary control device 700. In some examples, the user may use the external device 701 to inflate or deflate the cuff 704. For example, in response to the user activating an inflation cycle using the external device 701, the external device 701 may transmit a wireless signal to the pump device 706 to initiate the inflation cycle to transfer fluid from the fluid reservoir 702 to the cuff 704 (e.g., by opening an active valve where the pressure in the fluid reservoir 702 causes the fluid to move through the active valve to the cuff 704). In some examples, in response to the user activating a deflation cycle using the external device 701, the external device 701 may transmit a wireless signal to the pump device 706 to initiate the deflation cycle to transfer fluid from the cuff 704 to the fluid reservoir 702.

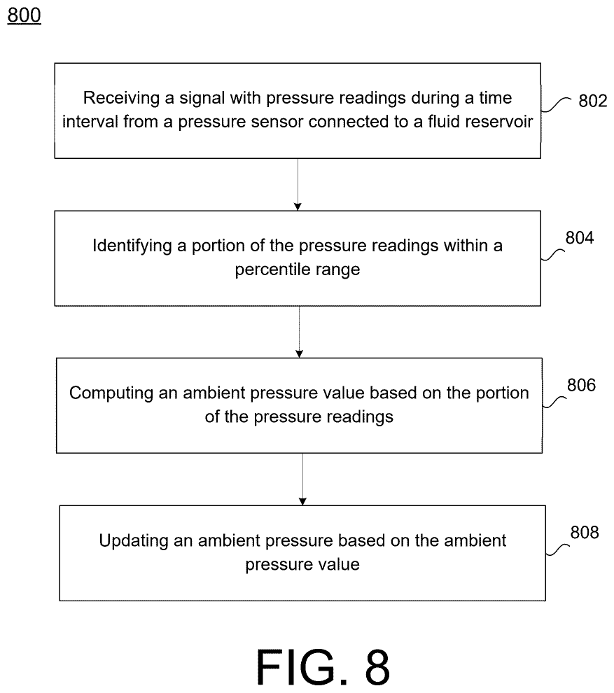

FIG. 8 illustrates a flowchart 800 depicting example operations of an electronic pump device for computing an ambient pressure value according to an aspect. Although the flowchart 800 of FIG. 8 illustrates the operations in sequential order, it will be appreciated that this is merely an example, and that additional or alternative operations may be included. Further, operations of FIG. 8 and related operations may be executed in a different order than that shown, or in a parallel or overlapping fashion.

Operation 802 includes receiving a signal with pressure readings during a time interval from a pressure sensor connected to a fluid reservoir. Operation 804 includes identifying a portion of the pressure readings within a percentile range. Operation 806 includes computing an ambient pressure value based on the portion of the pressure readings. Operation 808 includes updating an ambient pressure based on the ambient pressure value.

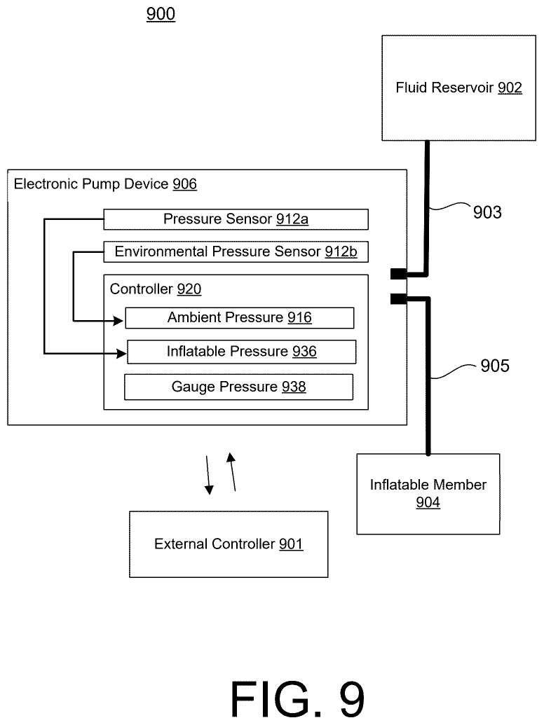

FIG. 9 illustrate an implantable medical device 900 with an electronic pump device 906 that includes an environmental pressure sensor 912b that monitors and computes ambient pressure 916 and uses the ambient pressure 916 in combination with an inflatable pressure 936, generated by a pressure sensor 912a, to compute a gauge pressure 938. The gauge pressure 938 is used to control the inflation and/or deflation of an inflatable member 904. In some examples, the implantable medical device 900 is an artificial urinary sphincter device. In some examples, the implantable medical device 900 is an inflatable penile prosthesis. However, the implantable medical device 900 may include any type of medical device that transfers fluid between components of the implantable medical device 900. The implantable medical device 900 may be an example of the implantable medical device 100 of FIGS. 1A to 1E and FIGS. 2 to 5 and may include any of the details discussed with reference to FIGS. 1A to 1E and FIGS. 2 to 5.

The implantable medical device 900 includes a fluid reservoir 902, an inflatable member 904, and an electronic pump device 906 configured to transfer fluid between the fluid reservoir 902 and the inflatable member 904. In some examples, the inflatable member 904 is an inflatable cuff member configured to be implemented around a urethra of a patient. In some examples, the inflatable member 904 is a penile prosthetic with one or more inflatable cylinders that may be implanted into the corpus cavernosum of the user. The fluid reservoir 902 may be implanted in the abdomen or pelvic cavity of the user (e.g., the fluid reservoir 902 may be implanted in the lower portion of the user's abdominal cavity or the upper portion of the user's pelvic cavity). In some examples, at least a portion of the electronic pump device 906 may be implemented in the patient's body.

The implantable medical device 900 may include a first tube member 903 and a second tube member 905. In some examples, the first tube member 903 and the second tube member 905 are referred to as conduit connectors. Each of the first tube member 903 and the second tube member 905 may define a lumen configured to transfer the fluid to and from the electronic pump device 906. The first tube member 903 may be coupled to the electronic pump device 906 and the fluid reservoir 902 such that fluid can be transferred between the electronic pump device 906 and the fluid reservoir 902 via the first tube member 903. The second tube member 905 may be coupled to the electronic pump device 906 and the inflatable member 904 such that fluid can be transferred between the electronic pump device 906 and the inflatable member 904 via the second tube member 905.

The electronic pump device 906 that can monitor control and regulate the pressure within an inflatable member 904. In some examples, the electronic pump device 906 is referred to as a can. The electronic pump device 906 may automatically transfer fluid between the fluid reservoir 902 and the inflatable member 904 without the user manually operating a pump (e.g., squeezing and releasing a pump bulb). The electronic pump device 906 includes pumps, valves, a battery, and electronic circuitry. The electronic pump device 906 may include an antenna configured to wirelessly transmit (and receive) wireless signals from an external device 901. The external device 901 may be any type of component that can communicate with the electronic pump device 906. The external device 901 may be a computer, smartphone, tablet, pendant, key fob, etc. A user may use the external device 901 to control the implantable medical device 900. In some examples, the user may use the external device 901 to inflate or deflate the inflatable member 904.

The electronic pump device 906 includes a plurality of pressure sensors. The electronic pump device 906 may include a pressure sensor 912a connected to the inflatable member 904. The pressure sensor 912a is used for measuring an inflatable pressure 936 of the inflatable member 904. The inflatable pressure 936 is the pressure inside of the inflatable member 904. In some examples, the pressure sensor 912a is included in a housing of the electronic pump device 906. In some examples, the pressure sensor 912a is coupled to a circuit substrate (e.g., the circuit substrate 210 of FIG. 2B). In some examples, the pressure sensor 912a is coupled to the fluidic manifold 208 of FIG. 2B. In some examples, the pressure sensor 912a is connected to a port (e.g., first port 276 of FIG. 2B or second port 278 of FIG. 2B) fluidly coupled to the inflatable member 904. In some examples, the pressure sensor 912a is coupled to (and, in some examples, inside of) the second tube member 905.

The electronic pump device 906 may include an environmental pressure sensor 912b configured to compute an ambient pressure 916. In some examples, the ambient pressure 916 is referred to as a local atmospheric pressure. In some examples, the environmental pressure sensor 912b is coupled to a circuit substrate (e.g., the circuit substrate 210 of FIG. 2B) inside a housing of the electronic pump device 906. In some examples, the environmental pressure sensor 912b is located within the electronic pump device 906, but not in line with the fluidic line to the reservoir 902. In some examples, the environmental pressure sensor 912b is not connected to a first port 276 of FIG. 2B or a second port 278 of FIG. 2B. In some examples, the environmental pressure sensor 912b is embedded into the external surface of the housing of the electronic pump device 906. In some examples, the environment pressure sensor 912b is coupled to the first sidewall 232 of FIG. 2B. In some examples, the environment pressure sensor 912b is coupled to the second sidewall 234 of FIG. 2B. In some examples, the environment pressure sensor 912b is coupled to the frame 240 of FIG. 2B. In some examples, the diaphragm of the environmental pressure sensor 912b may be positioned to face the external environment.

The electronic pump device 906 includes a controller 920 configured to compute the gauge pressure 938 based on the ambient pressure 916 and the inflatable pressure 936. In some examples, the controller 920 computes the gauge pressure 938 by adjusting the inflatable pressure 936 using the ambient pressure 916. In some examples, the controller 920 computes the gauge pressure 938 based on the difference between the ambient pressure 916 and the inflatable pressure 936. In some examples, the controller 920 computes an adjustment pressure using the ambient pressure 916 and adjusts the inflatable pressure 936 using the adjustment pressure. With use of the environmental pressure sensor 912b, the difference between the two pressure sensors can be used to determine a gauge pressure 938 within the inflatable member 904, thereby giving the patient repeatable performance of the device, regardless of their location.

In some examples, the controller 920 may use the value obtained from the environmental pressure sensor 912b to remove the component of the pressure reading from the pressure sensor 912a attributed to the ambient pressure 916 (e.g., the local atmospheric pressure) in order to obtain a gauge pressure 938 within the inflatable member 904. This compensated gauge pressure 938 can then be used to provide control of inflation of the inflatable member 904 in a predictable, reliable, and/or repeatable manner, regardless of location (e.g., elevation).

In some examples, the controller 920 may activate the environmental pressure sensor 912b to generate a signal with pressure readings during a time interval. The time interval may have a predetermined length (e.g., thirty seconds, one minute, two minutes, or three minutes, or generally any set length of time). In some examples, a time interval is referred to as a sampling period or a measurement period. Each pressure reading may include a value of the ambient pressure 916 and a timestamp. In some examples, the controller 920 may periodically activate the environmental pressure sensor 912b to generate the signal for a respective sampling period and determine whether to update the ambient pressure 916. In some examples, updating the ambient pressure 916 includes replacing an old value with a new value in a memory device of the electronic pump device 906. The controller 920 may activate the environmental pressure sensor 912b according to any of the techniques used for activating a pressure sensor 112a of FIGS. 1A to 5.

In some examples, the electronic pump device 906 includes an accelerometer (e.g., the accelerometer 154 of FIG. 4). In some examples, the accelerometer can be used to further improved the accuracy of the ambient pressure 916 (e.g., the local environmental pressure measurement) by determining the orientation of the patient. The orientation of the patient may have an impact on ambient pressure 916 and/or the inflatable pressure 936. The accelerometer could also be utilized for motion detection.

Clause 1. An implantable medical device comprising: an inflatable member; a fluid reservoir; a pressure sensor connected to the fluid reservoir; and an electronic pump device including a controller configured to execute operations, the operations comprising: receiving a signal with pressure readings during a time interval from the pressure sensor; identifying a portion of the pressure readings within a percentile range; computing an ambient pressure value based on the portion of the pressure readings; and updating an ambient pressure based on the ambient pressure value.

Clause 2. The implantable medical device of clause 1, wherein the pressure sensor is a first pressure sensor, the implantable medical device further comprising: a second pressure sensor connected to the inflatable member, wherein the operations further comprise: detecting an inflation pressure of the inflatable member using the second pressure sensor; computing a gauge pressure based on the ambient pressure and the inflation pressure; and controlling inflation or deflation of the inflatable member based on the gauge pressure.

Clause 3. The implantable medical device of clause 1 or 2, wherein the ambient pressure value is a first ambient pressure value, wherein the operations further comprise: detecting that a difference between the first ambient pressure value and a second ambient pressure value of a previous time interval is equal to or greater than a threshold level; and updating the ambient pressure with the first ambient pressure value.

Clause 4. The implantable medical device of any one of clauses 1 to 3, wherein the operations further comprise: computing the ambient pressure value by selecting a pressure reading with a minimum pressure value among the portion of the pressure readings.

Clause 5. The implantable medical device of any one of clauses 1 to 4, wherein the operations further comprise: computing a threshold deviation from a median using the portion of the pressure readings; determining whether a deviation of the ambient pressure value from the median is equal to or greater than the threshold deviation; in response to the deviation of the ambient pressure value from the median being equal to or greater than the threshold deviation, discarding the ambient pressure value; and in response to the deviation of the ambient pressure value from the median being less than the threshold deviation, accepting the ambient pressure value.

Clause 6. The implantable medical device of any one of clauses 1 to 5, wherein the operations further comprise: detecting a triggering event; and in response to detecting the triggering event, activating the pressure sensor to receive the pressure readings during the time interval.

Clause 7. The implantable medical device of any one of clauses 1 to 6, wherein the ambient pressure value is a first ambient pressure value, wherein the time interval is a first time interval, and the pressure readings are first pressure readings, wherein the operations further comprise: activating the pressure sensor to receive second pressure readings in a second time interval, the second time interval occurring before the first time interval, the second time interval having a length that is less than a length of the first time interval; computing a second ambient pressure value based on a portion of the second pressure readings that are within the percentile range; and in response to a difference between the second ambient pressure value and a previous ambient pressure value of a previous time interval being equal to or greater than a threshold level, activating the pressure sensor to receive the first pressure readings in the first time interval.

Clause 8. The implantable medical device of any one of clauses 1 to 7, wherein the operations further comprise: obtaining, from a memory device, a correction value relating to a user of the implantable medical device; and generating an updated ambient pressure based on the ambient pressure value and the correction value.

Clause 9. The implantable medical device of clause 8, wherein the operations further comprise: receiving acceleration data for one or more user positions during a calibration process; and generating the correction value based on the acceleration data.

Clause 10. The implantable medical device of any one of clauses 1 to 9, wherein the signal is a first signal, the first signal being generated according to a first sampling rate, wherein the operations further comprise: downsampling the first signal to generate a second signal with a second sampling rate, the second sampling rate being lower than the first sampling rate; and identifying the portion of the pressure readings within the percentile range from the second signal.

Clause 11. A method for measuring ambient pressure in an implantable medical device, the method comprising: receiving a signal with pressure readings during a time interval from a pressure sensor connected to a fluid reservoir; identifying a portion of the pressure readings within a percentile range; computing an ambient pressure value based on the portion of the pressure readings; and updating an ambient pressure based on the ambient pressure value.

Clause 12. The method of clause 11, wherein the pressure sensor is a first pressure sensor, the method further comprising: detecting an inflation pressure of an inflatable member using a second pressure sensor; computing a gauge pressure based on the ambient pressure and the inflation pressure; and controlling inflation or deflation of the inflatable member based on the gauge pressure.

Clause 13. The method of clause 11 or 12, wherein the ambient pressure value is a first ambient pressure value, the method further comprising: comparing the first ambient pressure value to a second ambient pressure value of a previous time interval; in response to a difference between the first ambient pressure value and the second ambient pressure value being equal to or greater than a threshold level, updating the ambient pressure with the first ambient pressure value; and in response to the difference between the first ambient pressure value and the second ambient pressure value being less than the threshold level, setting the ambient pressure as the second ambient pressure value.

Clause 14. The method of any one of clauses 11 to 13, further comprising: computing a threshold deviation from a median using the portion of the pressure readings; determining whether a deviation of the ambient pressure value from the median is equal to or greater than the threshold deviation; in response to the deviation of the ambient pressure value from the median being equal to or greater than the threshold deviation, discarding the ambient pressure value; and in response to the deviation of the ambient pressure value from the median being less than the threshold deviation, accepting the ambient pressure value.

Clause 15. The method of any one of clauses 11 to 14, wherein the signal is a first signal, the first signal being generated according to a first sampling rate, the method further comprising: downsampling the first signal to generate a second signal with a second sampling rate, the second sampling rate being lower than the first sampling rate; and identifying the portion of the pressure readings within the percentile range from the second signal.

Clause 16. An implantable medical device comprising: an inflatable member; a fluid reservoir; a pressure sensor connected to the fluid reservoir; and an electronic pump device including a controller configured to execute operations, the operations comprising: receiving a signal with pressure readings during a time interval from the pressure sensor; identifying a portion of the pressure readings within a percentile range; computing an ambient pressure value based on the portion of the pressure readings; and updating an ambient pressure based on the ambient pressure value.

Clause 17. The implantable medical device of clause 16, wherein the pressure sensor is a first pressure sensor, the implantable medical device further comprising: a second pressure sensor connected to the inflatable member, wherein the operations further comprise: detecting an inflation pressure of the inflatable member using the second pressure sensor; computing a gauge pressure based on the ambient pressure and the inflation pressure; and controlling inflation or deflation of the inflatable member based on the gauge pressure.

Clause 18. The implantable medical device of clause 16, wherein the ambient pressure value is a first ambient pressure value, wherein the operations further comprise: detecting that a difference between the first ambient pressure value and a second ambient pressure value of a previous time interval is equal to or greater than a threshold level; and updating the ambient pressure with the first ambient pressure value.

Clause 19. The implantable medical device of clause 16, wherein the operations further comprise: computing the ambient pressure value by selecting a pressure reading with a minimum pressure value among the portion of the pressure readings.

Clause 20. The implantable medical device of clause 16, wherein the operations further comprise: computing a threshold deviation from a median using the portion of the pressure readings; determining whether a deviation of the ambient pressure value from the median is equal to or greater than the threshold deviation; in response to the deviation of the ambient pressure value from the median being equal to or greater than the threshold deviation, discarding the ambient pressure value; and in response to the deviation of the ambient pressure value from the median being less than the threshold deviation, accepting the ambient pressure value.

Clause 21. The implantable medical device of clause 16, wherein the operations further comprise: detecting a triggering event; and in response to detecting the triggering event, activating the pressure sensor to receive the pressure readings during the time interval.

Clause 22. The implantable medical device of clause 16, wherein the ambient pressure value is a first ambient pressure value, wherein the time interval is a first time interval, and the pressure readings are first pressure readings, wherein the operations further comprise: activating the pressure sensor to receive second pressure readings in a second time interval, the second time interval occurring before the first time interval, the second time interval having a length that is less than a length of the first time interval; computing a second ambient pressure value based on a portion of the second pressure readings that are within the percentile range; and in response to a difference between the second ambient pressure value and a previous ambient pressure value of a previous time interval being equal to or greater than a threshold level, activating the pressure sensor to receive the first pressure readings in the first time interval.

Clause 23. The implantable medical device of clause 16, wherein the operations further comprise: obtaining, from a memory device, a correction value relating to a user of the implantable medical device; and generating an updated ambient pressure based on the ambient pressure value and the correction value.

Clause 24. The implantable medical device of clause 23, wherein the operations further comprise: receiving acceleration data for one or more user positions during a calibration process; and generating the correction value based on the acceleration data.

Clause 25. The implantable medical device of clause 16, wherein the signal is a first signal, the first signal being generated according to a first sampling rate, wherein the operations further comprise: downsampling the first signal to generate a second signal with a second sampling rate, the second sampling rate being lower than the first sampling rate; and identifying the portion of the pressure readings within the percentile range from the second signal.

Clause 26. A method for measuring ambient pressure in an implantable medical device, the method comprising: receiving a signal with pressure readings during a time interval from a pressure sensor connected to a fluid reservoir; identifying a portion of the pressure readings within a percentile range; computing an ambient pressure value based on the portion of the pressure readings; and updating an ambient pressure based on the ambient pressure value.

Clause 27. The method of clause 26, wherein the pressure sensor is a first pressure sensor, the method further comprising: detecting an inflation pressure of an inflatable member using a second pressure sensor; computing a gauge pressure based on the ambient pressure and the inflation pressure; and controlling inflation or deflation of the inflatable member based on the gauge pressure.

Clause 28. The method of clause 26, wherein the ambient pressure value is a first ambient pressure value, the method further comprising: comparing the first ambient pressure value to a second ambient pressure value of a previous time interval; in response to a difference between the first ambient pressure value and the second ambient pressure value being equal to or greater than a threshold level, updating the ambient pressure with the first ambient pressure value; and in response to the difference between the first ambient pressure value and the second ambient pressure value being less than the threshold level, setting the ambient pressure as the second ambient pressure value.

Clause 29. The method of clause 26, further comprising: computing a threshold deviation from a median using the portion of the pressure readings; determining whether a deviation of the ambient pressure value from the median is equal to or greater than the threshold deviation; in response to the deviation of the ambient pressure value from the median being equal to or greater than the threshold deviation, discarding the ambient pressure value; and in response to the deviation of the ambient pressure value from the median being less than the threshold deviation, accepting the ambient pressure value.

Clause 30. The method of clause 26, wherein the signal is a first signal, the first signal being generated according to a first sampling rate, the method further comprising: downsampling the first signal to generate a second signal with a second sampling rate, the second sampling rate being lower than the first sampling rate; and identifying the portion of the pressure readings within the percentile range from the second signal.

Clause 31. A non-transitory computer-readable medium storing executable instructions that cause at least one processor to execute operations, the operations comprising: receiving a signal with pressure readings during a time interval from a pressure sensor connected to a fluid reservoir, the fluid reservoir configured to be implanted in a body of a patient; identifying a portion of the pressure readings within a percentile range; computing an ambient pressure value based on the portion of the pressure readings; and updating an ambient pressure based on the ambient pressure value.

Clause 32. The non-transitory computer-readable medium of clause 31, wherein the pressure sensor is a first pressure sensor, wherein the operations further comprise: detecting an inflation pressure of an inflatable member using a second pressure sensor; computing a gauge pressure based on the ambient pressure and the inflation pressure; and controlling inflation or deflation of the inflatable member based on the gauge pressure.

Clause 33. The non-transitory computer-readable medium of clause 32, wherein the operations further comprise: computing the ambient pressure value by selecting a pressure reading with a minimum pressure value among the portion of the pressure readings.

Clause 34. The non-transitory computer-readable medium of clause 33, wherein the operations further comprise: detecting a triggering event; and in response to detecting the triggering event, activating the pressure sensor to receive the pressure readings during the time interval.

Clause 35. The non-transitory computer-readable medium of clause 34, wherein the ambient pressure value is a first ambient pressure value, wherein the time interval is a first time interval, and the pressure readings are first pressure readings, wherein the operations further comprise: activating the pressure sensor to receive second pressure readings in a second time interval, the second time interval occurring before the first time interval, the second time interval having a length that is less than a length of the first time interval; computing a second ambient pressure value based on a portion of the second pressure readings within the percentile range; and in response to a difference between the second ambient pressure value and a previous ambient pressure value of a previous time interval being equal to or greater than a threshold level, activating the pressure sensor to receive the first pressure readings in the first time interval.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable medical device comprising:
an inflatable member;
a fluid reservoir;
a pressure sensor connected to the fluid reservoir; and
an electronic pump device including a controller configured to execute operations, the operations comprising:
receiving a signal with pressure readings during a time interval from the pressure sensor;
identifying a portion of the pressure readings within a percentile range;
computing an ambient pressure value based on the portion of the pressure readings; and
updating an ambient pressure based on the ambient pressure value.

2. The implantable medical device of claim 1, wherein the pressure sensor is a first pressure sensor, the implantable medical device further comprising:
a second pressure sensor connected to the inflatable member, wherein the operations further comprise:
detecting an inflation pressure of the inflatable member using the second pressure sensor;
computing a gauge pressure based on the ambient pressure and the inflation pressure; and
controlling inflation or deflation of the inflatable member based on the gauge pressure.

3. The implantable medical device of claim 1, wherein the ambient pressure value is a first ambient pressure value, wherein the operations further comprise:
detecting that a difference between the first ambient pressure value and a second ambient pressure value of a previous time interval is equal to or greater than a threshold level; and
updating the ambient pressure with the first ambient pressure value.

4. The implantable medical device of claim 1, wherein the operations further comprise:
computing the ambient pressure value by selecting a pressure reading with a minimum pressure value among the portion of the pressure readings.

5. The implantable medical device of claim 1, wherein the operations further comprise:
computing a threshold deviation from a median using the portion of the pressure readings;
determining whether a deviation of the ambient pressure value from the median is equal to or greater than the threshold deviation;
in response to the deviation of the ambient pressure value from the median being equal to or greater than the threshold deviation, discarding the ambient pressure value; and
in response to the deviation of the ambient pressure value from the median being less than the threshold deviation, accepting the ambient pressure value.

6. The implantable medical device of claim 1, wherein the operations further comprise:
detecting a triggering event; and
in response to detecting the triggering event, activating the pressure sensor to receive the pressure readings during the time interval.

7. The implantable medical device of claim 1, wherein the ambient pressure value is a first ambient pressure value, wherein the time interval is a first time interval, and the pressure readings are first pressure readings, wherein the operations further comprise:
activating the pressure sensor to receive second pressure readings in a second time interval, the second time interval occurring before the first time interval, the second time interval having a length that is less than a length of the first time interval;
computing a second ambient pressure value based on a portion of the second pressure readings that are within the percentile range; and
in response to a difference between the second ambient pressure value and a previous ambient pressure value of a previous time interval being equal to or greater than a threshold level, activating the pressure sensor to receive the first pressure readings in the first time interval.

8. The implantable medical device of claim 1, wherein the operations further comprise:
obtaining, from a memory device, a correction value relating to a user of the implantable medical device; and
generating an updated ambient pressure based on the ambient pressure value and the correction value.

9. The implantable medical device of claim 8, wherein the operations further comprise:
receiving acceleration data for one or more user positions during a calibration process; and
generating the correction value based on the acceleration data.

10. The implantable medical device of claim 1, wherein the signal is a first signal, the first signal being generated according to a first sampling rate, wherein the operations further comprise:
downsampling the first signal to generate a second signal with a second sampling rate, the second sampling rate being lower than the first sampling rate; and
identifying the portion of the pressure readings within the percentile range from the second signal.

11. A method for measuring ambient pressure in an implantable medical device, the method comprising:
receiving a signal with pressure readings during a time interval from a pressure sensor connected to a fluid reservoir;
identifying a portion of the pressure readings within a percentile range;
computing an ambient pressure value based on the portion of the pressure readings; and
updating an ambient pressure based on the ambient pressure value.

12. The method of claim 11, wherein the pressure sensor is a first pressure sensor, the method further comprising:
detecting an inflation pressure of an inflatable member using a second pressure sensor;
computing a gauge pressure based on the ambient pressure and the inflation pressure; and
controlling inflation or deflation of the inflatable member based on the gauge pressure.

13. The method of claim 11, wherein the ambient pressure value is a first ambient pressure value, the method further comprising:
comparing the first ambient pressure value to a second ambient pressure value of a previous time interval;
in response to a difference between the first ambient pressure value and the second ambient pressure value being equal to or greater than a threshold level, updating the ambient pressure with the first ambient pressure value; and
in response to the difference between the first ambient pressure value and the second ambient pressure value being less than the threshold level, setting the ambient pressure as the second ambient pressure value.

14. The method of claim 11, further comprising:
computing a threshold deviation from a median using the portion of the pressure readings;
determining whether a deviation of the ambient pressure value from the median is equal to or greater than the threshold deviation;
in response to the deviation of the ambient pressure value from the median being equal to or greater than the threshold deviation, discarding the ambient pressure value; and
in response to the deviation of the ambient pressure value from the median being less than the threshold deviation, accepting the ambient pressure value.

15. The method of claim 11, wherein the signal is a first signal, the first signal being generated according to a first sampling rate, the method further comprising:
downsampling the first signal to generate a second signal with a second sampling rate, the second sampling rate being lower than the first sampling rate; and
identifying the portion of the pressure readings within the percentile range from the second signal.

16. A non-transitory computer-readable medium storing executable instructions that cause at least one processor to execute operations, the operations comprising:
receiving a signal with pressure readings during a time interval from a pressure sensor connected to a fluid reservoir, the fluid reservoir configured to be implanted in a body of a patient;
identifying a portion of the pressure readings within a percentile range;
computing an ambient pressure value based on the portion of the pressure readings; and
updating an ambient pressure based on the ambient pressure value.

17. The non-transitory computer-readable medium of claim 16, wherein the pressure sensor is a first pressure sensor, wherein the operations further comprise:
detecting an inflation pressure of an inflatable member using a second pressure sensor;
computing a gauge pressure based on the ambient pressure and the inflation pressure; and
controlling inflation or deflation of the inflatable member based on the gauge pressure.

18. The non-transitory computer-readable medium of claim 17, wherein the operations further comprise:
computing the ambient pressure value by selecting a pressure reading with a minimum pressure value among the portion of the pressure readings.

19. The non-transitory computer-readable medium of claim 18, wherein the operations further comprise:
detecting a triggering event; and
in response to detecting the triggering event, activating the pressure sensor to receive the pressure readings during the time interval.

20. The non-transitory computer-readable medium of claim 19, wherein the ambient pressure value is a first ambient pressure value, wherein the time interval is a first time interval, and the pressure readings are first pressure readings, wherein the operations further comprise:
activating the pressure sensor to receive second pressure readings in a second time interval, the second time interval occurring before the first time interval, the second time interval having a length that is less than a length of the first time interval;
computing a second ambient pressure value based on a portion of the second pressure readings within the percentile range; and
in response to a difference between the second ambient pressure value and a previous ambient pressure value of a previous time interval being equal to or greater than a threshold level, activating the pressure sensor to receive the first pressure readings in the first time interval.
